(19) United States
(12) Patent Application Publication
(10) Pub. No.: US 2026/0060939 A1
(43) Pub. Date: Mar. 5, 2026

(54) TREATMENT FOR MANAGEMENT AND PREVENTION OF EYELID CONDITIONS

(71) Applicants: John Olkowski, Honolulu, HI (US); Erika Olkowski, Honolulu, HI (US)

(72) Inventors: John Olkowski, Honolulu, HI (US); Erika Olkowski, Honolulu, HI (US)

(21) Appl. No.: 19/263,917

(22) Filed: Jul. 9, 2025

Related U.S. Application Data
(60) Provisional application No. 63/688,000, filed on Aug. 28, 2024.

(51) Int. Cl. A61K 31/05; A61K 9/00; A61K 31/00; A61K 36/534; A61K 36/61; A61K 36/752; A61P 27/02

(52) U.S. Cl. CPC A61K 31/05; A61K 9/0048; A61K 31/658; A61K 36/534; A61K 36/61; A61K 36/752; A61P 27/02

(57) ABSTRACT

Blepharitis, an infection and inflammation of the edge of the eyelids, and meibomian gland dysfunction (MGD), blockage of tiny oil glands at the eye lid margin, are quite common, usually under diagnosed and underappreciated diseases. There are number of causes for blepharitis, including bacterial components such as Staphylococcus aureus, Staphylococcus epidermidis, Propionibacterium acnes and Corynebacteria sp., seborrhea or microscopic mites (Demodex). More than 50% of patients seeking ophthalmologic consultations have symptoms and signs indicating one of these entities. Another large number of patients remain undiagnosed or in early stages of disease that could be easily treated thus preventing long-term pain and suffering. MGD is in fact the leading cause of dry eye disease, a potentially preventable malady.

### CROSS-REFERENCE TO RELATED APPLICATIONS

This application claims the benefit of U.S. Provisional Patent Application No. 63/688,000, filed on Aug. 24, 2024, which is incorporated by reference herein in its entirety

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants make no claim to any trademarks referenced.

### BACKGROUND OF THE INVENTION

### 1) Field of the Invention

The present disclosure relates to compositions for treating eyelid conditions, and more particularly to a carvacrol-based composition for managing and preventing blepharitis and other eyelid disorders. using chemical and mechanical treatments.

### 2) Description of Related Art

Blepharitis is a common, often chronic condition that can be challenging to treat, and affect any age group. Most of the time, blepharitis happens because you have too many bacteria on your eyelids and at the base of your eyelashes. Having bacteria on your skin is normal, but too many bacteria can cause problems. You can also get blepharitis if the oil glands in your eyelids get clogged or irritated. Blepharitis can lead to other ophthalmic diseases including chalazion, conjunctivitis, keratitis, and dry eyes. Bacteria can form a biofilm to protect itself from many current antibiotics.

Many patients may simultaneously suffer from blepharitis as a result of a Demodex mite infestation which presents with symptoms such as a foreign body sensation, redness and itching. The symptoms can become very severe. If Demodex mites are to be controlled on the eyelids, it must also be treated on the surrounding skin of the face or reinfestation of the eyelids will occur.

Unfortunately, no current single therapy can effectively and simultaneously treat these multiple eyelid conditions namely: 1) bacterial overgrowth with concomitant formation of a biofilm protective layer, 2) Demodex overgrowth on eyelids and surrounding facial area, 3) Seborrheic dermatitis and 4) active inflammation of the eyelids. Many products that may only treat one of these conditions can be extremely expensive and not affordable for most patients or may require treatment in the office under anesthesia.

Management of eyelid conditions typically involves a combination of hygiene practices, topical treatments, and in some cases, oral medications. However, existing treatment options may have limitations in terms of efficacy, patient compliance, or potential side effects with long-term use. For example, some topical antibiotics used to treat bacterial blepharitis can potentially lead to the development of resistant bacterial strains over time.

There is ongoing interest in exploring natural compounds with antimicrobial and anti-inflammatory properties for the management of eyelid conditions. Essential oils and their components have garnered attention due to their potential therapeutic effects. However, the application of such compounds near the sensitive eye area requires careful consideration of safety, tolerability, and efficacy.

As the prevalence of eyelid conditions continues to be a concern for both patients and healthcare providers, there is a need for innovative approaches that can address multiple aspects of these disorders, including microbial overgrowth, inflammation, and associated symptoms. Advancements in this field may contribute to improved management strategies and patient outcomes for individuals affected by eyelid conditions.

### BRIEF SUMMARY OF THE INVENTION

According to an aspect of the present disclosure, a composition for treating eyelid conditions is provided. The composition includes carvacrol and a therapeutic oil. The therapeutic oil is selected from the group consisting of an essential oil, a CBD oil, a cannabis oil, and other cannabis-derived oils. The carvacrol and the therapeutic oil are combined to form a mixture.

According to other aspects of the present disclosure, the composition includes the following features. The carvacrol is sonicated in the therapeutic oil. The composition is in a form selected from the group consisting of a cream, a serum, a gel, an ointment, and a lotion. The composition is formulated for topical application to an eyelid. The composition is effective for treating blepharitis. The composition is effective for treating at least one of: chalazion, stye, dry eye syndrome, eyelid dermatitis, and meibomian gland dysfunction. The carvacrol is present in a concentration range of 0.1% to 10% by weight of the composition. The therapeutic oil is present in a concentration range of 0.1% to 100% by weight of the composition.

According to another aspect of the present disclosure, a method of treating an eyelid condition is provided. The method includes applying a composition to an eyelid of a subject. The composition includes carvacrol and a therapeutic oil. The therapeutic oil is selected from the group consisting of an essential oil, a CBD oil, a cannabis oil, and other cannabis-derived oils. The carvacrol and the therapeutic oil are combined to form a mixture.

According to other aspects of the present disclosure, the method includes the following features. The eyelid condition is blepharitis. The eyelid condition is selected from the group consisting of chalazion, stye, dry eye syndrome, eyelid dermatitis, and meibomian gland dysfunction. The composition is applied topically to the eyelid. The composition is in a form selected from the group consisting of a cream, a serum, a gel, an ointment, and a lotion. The carvacrol is present in a concentration range of 0.1% to 10% by weight of the composition. The therapeutic oil is present in a concentration range of 0.1% to 100% by weight of the composition.

According to another aspect of the present disclosure, a kit for treating an eyelid condition is provided. The kit includes a composition and instructions for use. The composition includes carvacrol and a therapeutic oil. The therapeutic oil is selected from the group consisting of an essential oil, a CBD oil, a cannabis oil, and other cannabis-derived oils. The carvacrol and the therapeutic oil are combined to form a mixture.

According to other aspects of the present disclosure, the kit includes the following features. The composition is in a form selected from the group consisting of a cream, a serum, a gel, an ointment, and a lotion. The instructions include directions for applying the composition topically to an eyelid. The kit is effective for treating blepharitis. The kit is effective for treating at least one of: chalazion, stye, dry eye syndrome, eyelid dermatitis, and meibomian gland dysfunction. The carvacrol is present in a concentration range of 0.1% to 10% by weight of the composition. The therapeutic oil is present in a concentration range of 0.1% to 100% by weight of the composition.

Furthermore, the instant innovation in one form is directed to a single inexpensive topical treatment and prophylaxis of blepharitis, which has multiple causes namely bacteria, seborrhea and Demodex. The treatment has anti-inflammatory properties and a unique ability to eliminate biofilm. The treatment can also be used for other areas of infestation in humans and animals. The treatment treats multiple conditions that affect the eyelids including Blepharitis due to bacteria with its antibiotic properties and its ability to disrupt biofilm which can be the cause of antibiotic resistance and Blepharitis due to Demodex which affects almost 50% of the population. Carvacrol/Thymol is significantly more efficient at eradicating Demodex than Tea Tree Oil which is very popular. The treatment has anti-inflammatory properties to quiet irritation. This broad activity makes it an effective first line treatment for blepharitis with the potential to be used regularly at home for prophylaxis and treatment. The treatment can simultaneously treat multiple causes of eyelid disease and irritation with just one safe natural inexpensive product.

The instant innovation in one form is directed to a product for treating infectious and inflammatory eyelid disease with the product having properties to simultaneously resolve these major issues of the lid margin: Bacterial (with biofilm) blepharitis, seborrhea, Demodex blepharitis and inflammation. It is inexpensive, non-toxic and can be used long-term at home on a regular basis as a treatment as well as prophylaxis. This could help to alleviate many visits to the healthcare provider.

The composition includes a mixture of a vehicle including jojoba oil, Argan oil with active ingredients to include carvacrol and/or Thymol (from Oregano Oil, thyme oil or other similar oil).

The ability of carvacrol to cause leakage of cellular contents suggests that its action may cause pores in the bacterial membranes.

Carvacrol (C10H14O) has also been reported to be safe and exert minimal toxicity on human cells. This bacterial selectivity might be attributed to different physicochemical properties of membrane components, especially phospholipids found in bacteria versus mammalian cells. Cholesterol is the major lipid component of the eukaryotic membrane but not of the bacterial cell membrane and may lead to discrimination between bacterial and host cell membranes.

Since carvacrol is listed as a Generally Recognized as Safe (GRAS) food additive by the United States Food and Drug Administration, a significant potential exists for carvacrol as a safe molecule for broader therapeutic applications.

Carvacrol and thymol, alone or in oregano oil, could diffuse through the polysaccharide matrix of the biofilm and destabilize it thereby forming an antibiotic head. Thyme oil as a source of carvacrol. Although Oregano generally has a much higher concentration (80-90%), Thyme is second at (5-60%). The use of gamma-terpinene has also been explored and Carvacrol is a bio synthesized from gamma-terpinene in plants. Carvacrol as an antimicrobial has the following beneficial attributes:
Bacteria inhibition growth (liquid phase)
Gram-positive
Gram-negative
Oral Pathogen
Bacteria inhibition growth in vapor phase
Spores
Fungi inhibition growth
Inhibition of biofilm

According to another aspect of the present disclosure, a composition for treating eyelid conditions is provided. The composition includes carvacrol and a therapeutic oil. The carvacrol and the therapeutic oil are combined to form a mixture. The therapeutic oil is selected from the group consisting of an essential oil, oregano oil, thyme oil, flax seed oil, silicone oil, olive oil, peanut oil cannabidiol a CBD oil, a cannabis oil or other cannabis-derived oil and other natural oils or any combination thereof.

According to another aspects of the present disclosure, the composition may be combined by sonication or other high shear mixing methods. The eyelid condition may be blepharitis or other disease of the meibomian gland.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 is a table showing antimicrobial activity of carvacrol against strains of microorganisms, according to aspects of the present disclosure;

FIG. 2 is a chart comparing carvacrol containing various essential oils, according to aspects of the present disclosure;

FIG. 3 shows a GC/MS analysis of Melaleuca alternifolia L. essential oil, according to aspects of the present disclosure;

FIG. 4 shows a GC/MS analysis of Thymus serpyllum L. essential oil, according to aspects of the present disclosure;

FIG. 5 shows a table of the effects of selected essential and carrier oils on survival time of Demodex species, according to aspects of the present disclosure;

FIG. 6 shows forming a mixture of an antimicrobial agent using carvacrol, according to an embodiment;

FIG. 7 shows a glass beaker containing an antimicrobial agent using carvacrol in an infused oil, according to aspects of the present disclosure;

FIG. 8 shows carvacrol in an infused oil applied to a swab to treat the eyelid margins of an eye;

FIG. 9 shows carvacrol in an infused oil being applied to an eye using an eye dropper;

FIG. 10 shows carvacrol in an infused oil being applied to an eye using an eye dropper vial;

FIG. 11 shows forming an alternative mixture of an antimicrobial agent using carvacrol; and

FIG. 12 shows an alternative antimicrobial agent using carvacrol in an infused oil.

FIG. 13 shows a kit comprised of carvacrol and a therapeutic oil selected from the group consisting of an essential oil, a CBD oil, a cannabis oil, and other cannabis-derived oils, and instructions for use, eye dropper vial and a swab.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

Bacteria and Biofilm-Even though blepharitis is one of the most frequently observed conditions, with some form of blepharitis present in 15 to 25 percent of the population, it is also one of the most frequently mistreated due to a plethora of possible etiologies and symptoms that overlap with other ocular surface diseases. Signs and symptoms of blepharitis include swelling, thickening, scaling and/or crusting of the eyelids, redness of the lid margins, bulbar and palpebral hyperemia, gritty eyes and itchy eyelids.

For example, a recent survey concluded that 34 million adults experienced at least one of these symptoms most commonly associated with blepharitis in the past year, yet only 4.5 million people have received treatment for their blepharitis.

Bacteria colonize the lid margin within a structure known as a biofilm. The biofilm allows for population densities that initiate quorum-sensing gene activation. These newly activated gene products consist of inflammatory virulence factors, such as exotoxins, cytolytic toxins, and super-antigens, which are then present for the rest of the patient's life. The biofilm never goes away; it only thickens with age, producing increasing quantities of bacterial virulence factors, and thus, increasing inflammation. These virulence factors are likely the culprits that first cause inflammation, then meibomian gland dysfunction, aqueous insufficiency dry eye disease, and finally, lid destruction.

Many studies have been conducted to identify the nature of preoperative normal conjunctival flora; they have shown the existence of both pathogenic and nonpathogenic bacteria, with S. aureus being the most frequent pathogen recovered. Even for nonpathogenic bacteria like S. epidermidis, increased colony density leads to quorum sensing and can cause these bacteria to undergo a genetic shift and produce inflammatory mediators. The dearth of definitive treatments combined with the physicians' need for one creates a sense of urgency in finding an appropriate treatment.

Traditional treatment for bacterial blepharitis includes topical antibiotics including bacitracin, erythromycin and tobramycin. These antibiotics have the potential to develop resistant strains of bacteria and indeed may not be able to penetrate the protective biofilm produced by these bacteria. Biofilm cells can become up to 100-1000 times more resistant to antimicrobial substances than non-biofilm cells.

The present disclosure relates to a composition for treating eyelid conditions. The composition includes carvacrol and a therapeutic oil. The therapeutic oil includes an essential oil, a cannabidiol (CBD) oil, or other cannabis-derived oil. The composition provides an effective treatment for various eyelid conditions while offering potential benefits over existing treatments

As a monoterpenoid phenol, carvacrol is a component of many essential oils and is usually found in plants together with its isomer, thymol. Carvacrol, either alone or in combination with other compounds, has a strong antimicrobial effect on many different strains of bacteria and fungi that are dangerous to humans or can cause significant losses in the economy. Carvacrol also exerts strong anti-inflammatory properties by preventing the peroxidation of polyunsaturated fatty acids by inducing SOD, GPx, GR, and CAT, as well as reducing the level of pro-inflammatory cytokines in the body.

Carvacrol (5-isopropyl-2-methylphenol) is a monoterpenoid alcohol, a liquid with a boiling point of 236-237° C. and a density of 0.976 g/mL at 20° C. It is insoluble in water but very soluble in ethanol, acetone, and diethyl ether. It is a component of essential oils obtained from many plants, e.g., Corido thymus, Lippia pepperwort, black cumin (Nigella sativa), oregano (Origanum compactum), O. dictamnus, O. microphyllum, O. onites, O. scabrum, O. vulgare, pepperwort (Lepidium flavum), wild bergamot (Citrus aurantium var. bergamia Loisel), Monarda didyma, thyme (Thymus glandulosus), and savory (Satureja hortensis).

Carvacrol has been approved by the FDA for use in food and listed by the Council of Europe as a Category B chemical flavoring agent that may be added to foodstuffs at a level of 2 ppm in beverages, 5 ppm in flakes, and 25 ppm in candies.

In dental practice, carvacrol has been used as a substitute for cretol and carbolic acid in the treatment of toothache, sensitive dentine, and alveolar abscess, and as an antiseptic in the pulp canals of the teeth.

The mechanism of carvacrol is postulated as a modification of active sites on bacterial cells, inhibition of enzymes that catalyze or degradate antibiotics, inhibition of efflux pumps, or increase of membrane permeability. The ability of carvacrol to cause leakage of cellular contents suggests that its action may cause pores in the bacterial membranes. It is hypothesized that carvacrol causes Cell lysis which is the process of breaking down a cell's outer membrane, or plasma membrane, to release its contents thereby resulting in death of the bacteria causing the inflammation.

Carvacrol was found to alter cell membrane permeability, causing nucleic acids and proteins to leak and resulting in cell death. This mechanism was confirmed by the observation of alkaline phosphatase (AKP) leakage. This is an intracellular enzyme located between the cell wall and the cell membrane, and in the event of damage to the cell wall, there is a strong leakage of AKP outside the bacteria into the extracellular environment.

Carvacrol has been reported to be safe and exert minimal toxicity on human cells. This bacterial selectivity might be attributed to different physicochemical properties of membrane components, especially phospholipids found in bacteria versus mammalian cells. Cholesterol is the major lipid component of the eukaryotic membrane but not of the bacterial cell membrane and may lead to discrimination between bacterial and host cell membranes.

Since carvacrol is listed as a Generally Recognized as Safe (GRAS) food additive by the United States Food and Drug Administration, a significant potential exists for carvacrol as a safe molecule for broader therapeutic applications. Carvacrol has been approved by the FDA for use in food and listed by the Council of Europe as a Category B chemical flavoring agent that may be added to foodstuffs at a level of 2 ppm in beverages, 5 ppm in flakes, and 25 ppm in candies. In dental practice, carvacrol has been used as a substitute for cretol and carbolic acid in the treatment of toothache, sensitive dentine, and alveolar abscess, and as an antiseptic in the pulp canals of the teeth.

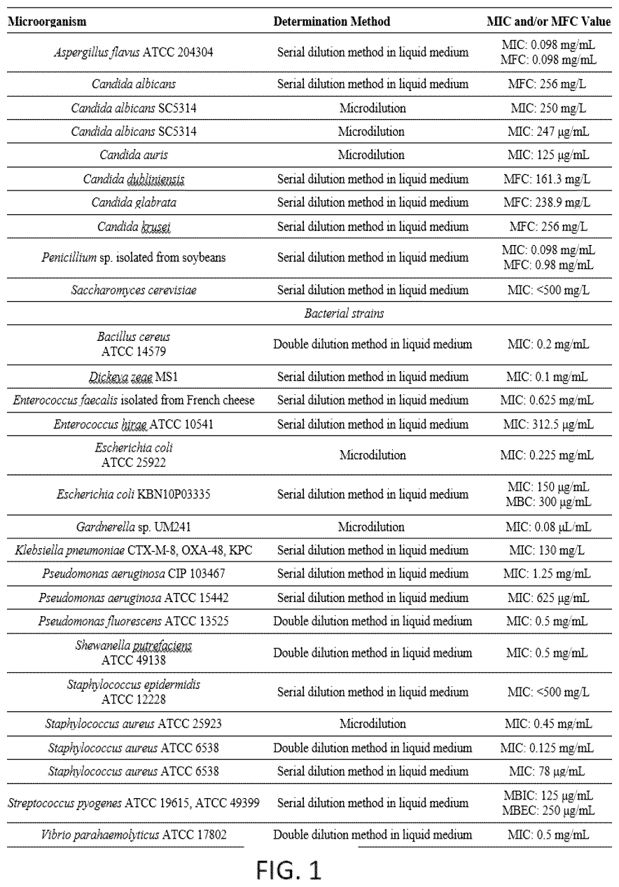

Carvacrol has a strong antimicrobial effect against many strains of bacteria and fungi that are dangerous to humans or cause significant losses in the economy. FIG. 1 shows a table of antimicrobial activity of carvacrol against strains of microorganisms.

The antimicrobial efficacy of these essential oils is demonstrated in FIG. 1, which presents a table of antimicrobial activity against various microorganisms. The table shows minimum inhibitory concentration (MIC) and minimum fungicidal concentration (MFC) values for different strains of bacteria and fungi. These values indicate the effectiveness of the essential oil components in inhibiting microbial growth.

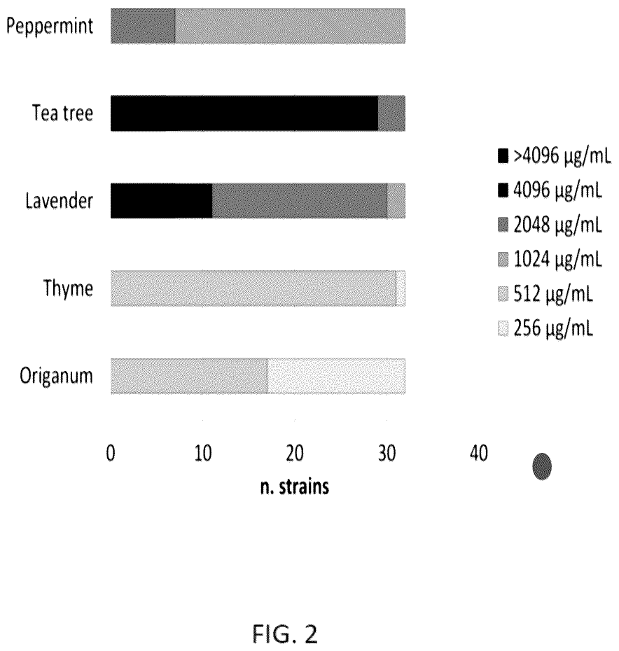

The carvacrol oil mixture demonstrates efficacy in treating various ocular conditions beyond eyelid disorders. The antimicrobial properties of the composition, as illustrated in FIG. 1 and FIG. 2, indicate potential applications for treating conjunctivitis, keratitis, and other microbial eye infections. The broad-spectrum activity against fungi and bacteria suggests the carvacrol oil mixture as an alternative to conventional antibiotic treatments, potentially reducing the risk of antibiotic resistance.

The anti-inflammatory properties of the carvacrol oil mixture contribute to its effectiveness in managing chronic ocular surface diseases. The composition addresses both the infectious and inflammatory components of conditions such as dry eye syndrome and meibomian gland dysfunction. This dual action provides a comprehensive approach to ocular surface health.

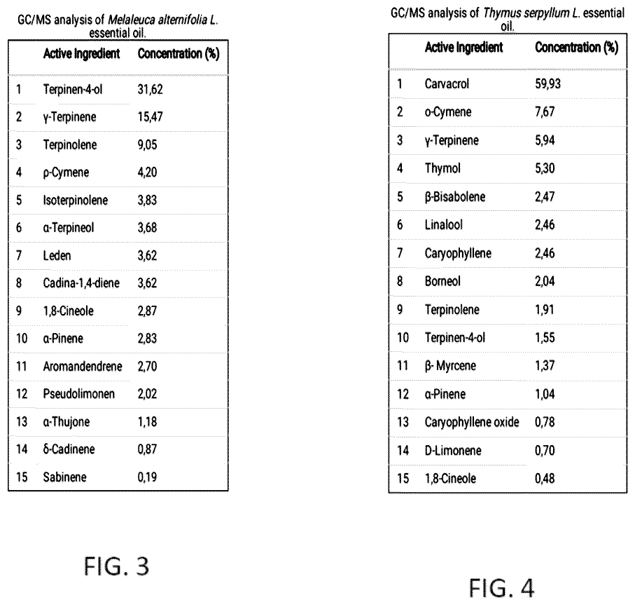

Long-term use of the carvacrol oil mixture offers advantages over existing treatments. Unlike topical steroids, which can lead to complications with prolonged use, the carvacrol oil mixture does not carry the same risks of inducing glaucoma or cataracts. The natural origin of the components, as evidenced by the GC/MS analyses in FIG. 3 and FIG. 4, suggests a lower likelihood of adverse reactions compared to synthetic pharmaceuticals.

In particular, carvacrol has extensively been tested as an antimicrobial agent in food to control Gram-positive and Gram-negative pathogens, including Bacillus cereus, Enterococcus faecalis, Listeria monocytogenes, Staphylococcus aureus, Escherichia coli, Pseudomonas fluorescens, Salmonella typhimurium, Vibrio cholerae, and V. vulnificus.

In another study, the antimicrobial activity of five essential oils from different plants was tested against pharyngeal erythromycin-resistant group A strep isolates, all erythromycin-resistant and cell-invasive strains. The antimicrobial activity of thyme and Origanum oils was high, with MICs much lower than those of lavender, mint, and tea tree oils. Since carvacrol is a major component of thyme and Origanum essential oils, the susceptibility of GAS strains to carvacrol was tested. Findings demonstrated a high activity of carvacrol against GAS, with MICs lower than those reported for other Gram-positive bacteria.

Carvacrol containing essential oils (oregano and thyme) had the highest antimicrobial activity (compared to tea tree oil (TTO) which had the worst antimicrobial activity) as shown in the table of FIG. 2.

The composition demonstrates antimicrobial activity against various microorganisms, as illustrated in FIG. 1. FIG. 2 which compares the antimicrobial activity of different essential oils including peppermint, tea tree, lavender, thyme, and Origanum oil and the activity of the composition against various microorganisms is demonstrated can also be found in FIG. 1. FIG. 2 which compares the antimicrobial activity of different essential oils, including tea tree, thyme, and oregano oils. The chart illustrates that thyme and Origanum oils, which contain high concentrations of carvacrol, exhibit superior antimicrobial activity compared to other essential oils. These oils are effective against a larger number of strains at lower concentrations, indicating their potential for treating eyelid conditions caused by microbial infections. These oils are effective against a larger number of strains at lower concentrations.

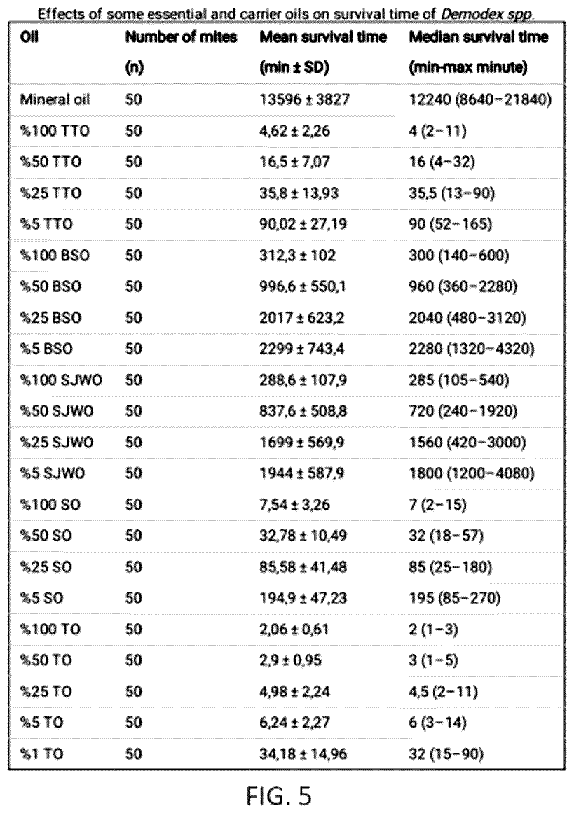

FIG. 3 and FIG. 4 provide GC/MS analyses of Melaleuca alternifolia L. and Thymus serpyllum L. essential oils, respectively. FIG. 5 presents data on the effects of selected essential and carrier oils on survival time of Demodex species, indicating the potential efficacy of the composition against these mites. The table demonstrates that higher concentrations of essential oils significantly reduce the survival time of Demodex mites compared to mineral oil.

As can be seen from FIG. 4 the GC/MS analysis of Thymus serpyllum L. essential oil, or thyme oil. The analysis shows that carvacrol is the dominant active ingredient, constituting 59.93% of the oil. Other notable components include o-Cymene (7.67%), γ-Terpinene (5.94%), and thymol (5.30%). The high concentration of carvacrol in thyme oil contributes to its potent antimicrobial activity.

The essential oils used in the composition for treating eyelid conditions contain various active ingredients that contribute to their therapeutic effects. FIG. 3 illustrates a GC/MS analysis of Melaleuca alternifolia L. essential oil, commonly known as tea tree oil. The analysis reveals that Terpinen-4-ol is the primary component, comprising 31.62% of the oil. Other significant components include γ-Terpinene (15.47%), Terpinolene (9.05%), and p-Cymene (4.20%). These compounds contribute to the antimicrobial properties of tea tree oil.

The GC/MS analysis of Melaleuca alternifolia L. essential oil, shown in FIG. 3, reveals the presence of various active ingredients. While Terpinen-4-ol is the primary component at 31.62%, other compounds contribute to the overall antimicrobial properties of the oil. FIG. 4 presents the GC/MS analysis of Thymus serpyllum L. essential oil, highlighting carvacrol as the dominant active ingredient at 59.93%. This high concentration of carvacrol in thyme oil explains its strong antimicrobial activity observed in FIG. 2.

The antimicrobial properties of the composition extend to its effectiveness against Demodex mites, which are often associated with eyelid conditions such as blepharitis. FIG. 5 presents data on the effects of various essential and other carrier oils on the survival time of Demodex species. The table demonstrates that higher concentrations of essential oils, particularly those containing carvacrol, significantly reduce the survival time of Demodex mites compared to mineral oil.

The data in FIG. 5 indicates that 100% concentrations of Tea Tree Oil (TTO), Black Seed Oil (BSO), and St. John's Wort Oil (SJWO) are effective in reducing the survival time of Demodex mites. However, the most notable results are observed with Thyme Oil (TO). At a 5% concentration, TO demonstrates a mean survival time of 10.8 minutes, which is significantly shorter than other oils at higher concentrations.

The effectiveness of TO against Demodex mites aligns with the GC/MS analysis presented in FIG. 4. The analysis of Thymus serpyllum L. essential oil reveals carvacrol as the primary active ingredient, comprising 59.93% of the oil. This high concentration of carvacrol contributes to the potent anti-Demodex activity observed in the survival time data.

Furthermore, the inclusion of cannabidiol (CBD) or other cannabis-derived oils in the carvacrol oil mixture enhances the therapeutic properties of the composition. This combination provides a comprehensive approach to treating eyelid conditions, addressing multiple symptoms and underlying causes.

The composition includes carvacrol and a therapeutic oil as its main components. Carvacrol is a monoterpenoid phenol with antimicrobial properties. The therapeutic oil includes essential oils, cannabidiol (CBD) oil, or other cannabis-derived oils.

Examples of therapeutic oils include oregano oil, thyme oil, cannabidiol (CBD) oil and tea tree oil. Oregano oil is derived from the Origanum vulgare plant and contains high levels of carvacrol. Thyme oil, obtained from Thymus vulgaris, also contains significant amounts of carvacrol and thymol. Tea tree oil, extracted from Melaleuca alternifolia, has antimicrobial properties.

When biofilm formation is analyzed one can see that it is formed by microbial cells that are irreversibly bound to a substrate or interface, or to each other. They are encapsulated in the extracellular polymeric substance (EPS) matrix which they produce, and exhibit an altered phenotype in terms of growth rate and gene transcription. In the studies of Wijesundar et al., it was found that carvacrol induced deformations and damage to the bacterial cells, as well as deactivation of extracellular polymeric substance (EPS), which, in the bacterial cell, are responsible for protecting the cell against toxic substances and ensuring its strength. In addition, carvacrol reduced the hydrophobic properties of multicellular bacterial structures by up to 84.2%, and was able to reduce the expression of the LuxS gene associated with the formation of biofilms by S. pyogene. Carvacrol also resulted to be a promising antibacterial agent against S. aureus and S. epidermidis, which mainly cause diseases of the skin and the respiratory, urinary, and digestive systems, as well as osteomyelitis of the bone and meningitis. In a study by Mauriello et al.

On the surface of materials commonly used in medicine and food production, a microbial biofilm often develops which is difficult to remove and promotes the development of pathogenic bacteria. Carvacrol inhibited biofilm formation by up to 74-88% for Pseudomonas aeruginosa ATCC15442, and up to 86-100% for S. aureus ATCC6538.

In this study, 79 essential oils were screened for antibiofilm ability against uropathogenic E. coli (UPEC). Components of active oils were identified, and their antibiofilm activities were also investigated using 96-well plates with crystal violet assay, scanning electron microscopy, and confocal laser scanning microscopy. Oregano oil and thyme red oil and their major common constituents, carvacrol and thymol, significantly inhibited UPEC biofilm formation at subinhibitory concentrations (<0.01%). These findings were supported by observations that carvacrol and thymol reduced fimbriae production and the swarming motility of UPEC. Furthermore, carvacrol and thymol markedly decreased the hemagglutinating ability of UPEC, and UPEC was more easily killed by human whole blood in the presence of carvacrol and thymol. In the wake of rising antimicrobial resistance, it is shown that carvacrol and thymol could be used to prevent biofilm formation by UPEC and to reduce its virulence.

Carvacrol possessed a strong inhibitory effect on the biofilm in the early stage. The mature biofilm often makes it easier to counteract antibiotics because of the protective effect of its physical barrier and anti-immune clearance. The results on scavenging effects of carvacrol showed that carvacrol at 1×MIC concentration cleared up to 93.75% of the mature biofilm, indicating that carvacrol could penetrate and destroy mature biofilms. This makes carvacrol have good antibacterial effects.

Another study evaluated the effect of oregano essential oil, carvacrol and thymol on biofilm-grown Staphylococcus aureus and Staphylococcus epidermidis strains, as well as the effects of the oils on biofilm formation. For most of the S. aureus (n=6) and S. epidermidis (n=6) strains tested, the biofilm inhibitory concentration (0.125-0.500%, v/v, for oregano, and 0.031-0.125%, v/v, for carvacrol and thymol) and biofilm eradication concentration (0.25-1.0%, v/v, for oregano and 0.125-0.500%, v/v, for carvacrol and thymol) values were twofold or fourfold greater than the concentration required to inhibit planktonic growth. Subinhibitory concentrations of the oils attenuated biofilm formation of S. aureus and S. epidermidis strains on polystyrene microtitre plates.

The virulence of Staphylococcus spp. predominantly found on the ocular surface has been attributed to their ability to form a biofilm which confers antimicrobial resistance. This study confirms that ocular Staphylococcus aureus and S. epidermidis collected from the cornea, conjunctiva, eyelid margin, intraorbital foreign body, intraocular lenses, vitreous and aqueous humors of corneal ulcer patients and from patients with other ocular diseases including endophthalmitis exhibit biofilm-forming capacity.

Another solution was to incorporate carvacrol and carboxybetaine into the polyurethane, slowing down the release of carvacrol from the material. A synergistic antibacterial effect of bio-derived “kill and gun” polyurethane against E. coli and S. aureus was observed.

Very recently published research on clove and thyme oils efficiency against Fusarium spp. development on soft contact lenses indicated that at concentrations lower than 50 μL/L that can be used without affecting the device material, the fungal cell adherence and formation were inhibited.

A contact lens impregnated or treated with a carvacrol solution may be used to prevent ocular infections and or treat existing ones. The incorporation of the carvacrol oil mixture into contact lenses offers several advantages. First, it ensures consistent delivery of the therapeutic components directly to the ocular surface. Second, it combines vision correction with treatment, improving patient compliance. Third, it provides a barrier against potential pathogens, reducing the risk of contact lens-associated infections.

The process of incorporating the carvacrol oil mixture into contact lenses involves impregnating the lens material with the composition during the manufacturing process. Alternatively, the contact lenses are soaked in the carvacrol oil mixture before use, allowing the lens material to absorb the active components.

The carvacrol-infused contact lenses are particularly beneficial for patients with chronic ocular surface diseases or those at high risk of eye infections. The sustained release of the antimicrobial and anti-inflammatory components of the carvacrol oil mixture provides continuous protection and treatment throughout the day.

The combination of the carvacrol oil mixture with contact lens technology represents a significant advancement in ocular therapeutics. This approach bridges the gap between traditional topical treatments and sustained-release drug delivery systems, offering a novel solution for managing various ocular conditions.

Regular use of the essential oils of oregano and thyme, and its active ingredients, carvacrol and thymol may prove to be invaluable in the prevention and treatment of bacterial blepharitis. They display broad antimicrobial properties. In addition, the efficacy they have against biofilm formation and elimination, which is unheard of in any currently available product, is an argument for its uniqueness and advantages in this arena. In addition, incorporating these ingredients into contact lens material may decrease the incidence of contact lens induced infections. Also using a carvacrol impregnated contact lens may be used to treat existing ocular infections. Carvacrol may also be used in contact lens solutions or impregnated into contact lens materials to prevent contact lens related eye infections. Carvacrol may be used as a preoperative treatment to prevent infections during ocular or periocular surgery and when coupled with cannabidiol (CBD) oil it can also provide an anti-inflammation treatment.

Demodex mites, Demodex folliculorum and Demodex brevis are microscopic, obligate, elongated mites that are the most common permanent intracutaneous parasites inhabiting the hair follicles and sebaceous glands of humans and animals. Mating takes place in the follicle opening and eggs are laid inside the hair follicles or sebaceous glands. The six-legged larvae hatch after 3-4 days, and the larvae develop into adults in about 7 days. Demodex has a life cycle of about 14 days. The total lifespan of a Demodex mite is several weeks. The dead mites decompose inside the hair follicles or sebaceous glands.

Demodex can be found on the face, including cheeks, nose, chin, forehead, temples, eye lashes, brows, and also on the scalp, neck, and ears. Other seborrheic locations such as naso-labial folds, peri-orbital areas, and less commonly upper and medial region of chest and back can also be infested.

The recent Trattler study reported that 57.7% of patients on exam for blepharitis had Demodex mites.

Another group sampled individuals using traditional approaches which revealed 14% of individuals over the age of 18 had visually observed mites. But when they evaluated the presence of mites based on the amplification of Demodex DNA, they found that every adult over 18 years of age and 70% of 18-year-olds had detectable Demodex 16S rDNA in the collected sebum of facial samples.

In some cases, a mite density of greater than 5 mites/cm2 in the pilo-sebaceous unit or 5 or more mites per follicle correlates with a Demodex infestation.

Among a wide range of reported species, only two, Demodex folliculorum and Demodex brevis, are found to parasitize the human body surface. Demodex folliculorum has been found for example on eyelash follicles, while Demodex brevis has been found, for example, proximate meibomian (tarsal) glands around the eye and sebaceous glands of the skin. Meibomian glands are a holocrine type of exocrine glands, at the rim of the eyelids inside the tarsal plate, responsible for the supply of meibum, an oily substance that prevents evaporation of the eye's tear film. Meibum prevents tear spillage onto the cheek, trapping tears between the oiled edge and the eyeball, and makes the closed lids airtight. There are approximately 50 glands on the upper eyelids and 25 glands on the lower eyelids. Symbiotic bacteria on the mites also can contribute to pathology. Increased sebum secretion and an increased number of sebaceous glands can provide a favorable habitat for the mites. While some level of Demodex can be asymptomatic, multiplication of Demodex mites to high densities, and/or a concurrent immune imbalance usually leads to skin damage. A growing body of literature implicates Demodex mites in anterior and posterior blepharitis. For example, Demodex has been implicated in 45% of blepharitis cases. It has been estimated that the prevalence of ocular surface disease is about 30 million patients; 19 million of which have Meibomian gland dysfunction/posterior blepharitis; 9 million with Demodex infestation, and 4 million with clear signs of Demodex.

Although it commonly causes skin problems, it also plays a role in the etiology of ocular diseases such as blepharitis and chalazion in the eyelid. As a general rule, ocular demodicosis treatment takes several months. The conventional treatment with daily lid scrub using Baby Shampoo could not kill Demodex. The use of yellow mercury ointment (mercury oxide ointment), sulfur ointment, camphor oil, crotamitone, choline esterase inhibitors, sulfacetamide, steroids, antibiotics and antimycotic drugs provides some improvements (Czepita et al. 2007). Permethrin or metronidazole has been applied topically in the treatment of blepharitis associated with Demodex spp. Success has been reported with oral ivermectin.

Currently a recommended treatment is the application of tea tree oil (TTO) containing terpinen-4-ol (T40) (Zorbozan et al. 2016). There are studies indicating successful treatment with TTO with 50% TTO and at home TTO shampoo in patients with ocular demodicosis (Kim et al. 2011; Gao et al. 2012; Cheng et al. 2015; Hirsch-Hoffmann et al. 2015). However current effective treatment with TTO requires 50% TTO which is irritating to the eye and requires treatment be done by practitioner in office under anesthesia to avoid irritation to the eye. Also, it has been reported that up to 3.5% of patients develop allergic reactions after the use of TTO and ocular irritation occurs as a treatment complication (Sȩdzikowska et al. 2015). Of all essential oils, TTO has caused the most (published) allergic reactions since the first cases were reported in 1991. In routine testing, prevalences of positive patch test reactions have ranged from 0.1% to 3.5%.

More recently Lotilaner 0.25% (Xdemvy®) was the first FDA approved treatment for Demodex blepharitis. This composition of Lotilanor eye drops has shown a mite eradication of only 67.9% of patients after a complete 43-day treatment course. This, however, does not address the issues of Demodex mites on the surrounding skin, which would reinhabit the lid margins. In addition, no long-term data on the efficacy of this product on the eyelids has been shown. Lastly the cost of this product without insurance is approximately $1800 a bottle while even with insurance the cost is approximately $200/bottle.

Herein is described a composition of essential oils containing carvacrol and thymol to treat Demodex mites.

FIG. 3 shows a GC/MS analysis of Melaleuca alternifolia L., revealing its major components. FIG. 4 presents a similar analysis for Thymus serpyllum L. essential oil, highlighting carvacrol as the primary active ingredient at 59.93%.

The ratios and concentrations of carvacrol and therapeutic oil in the composition vary. In some cases, the concentration of carvacrol is within a range of 0.1% to 10% by weight of the composition. The concentration of therapeutic oil is within a range of 0.1% to 100% by weight of the composition.

FIG. 5 shows a table of the effects of selected essential and carrier oils on survival time of Demodex spp. As supported in FIGS. 5, the mite killing time of 5% Thyme oil (TO) containing carvacrol and Thymol was significantly shorter than all other oil concentrations except 100% Tea Tree Oil (TTO). It was determined that 1% TO had a more effective killing time than 5% concentrations of TTO, a significant difference was found. It is thought that an essential oil, which has a high total carvacrol and thymol content of can be used for medical purposes.

Acarine arachnids such as the Demodex mite, Demodex folliculorum, also can infest mammalian quadrupeds, in particular domestic animals, especially dogs, causing demodetic mange. The Demodex mite burrows into hair follicles and sebaceous glands of the animal, the mite often causes severe dermatitis, infection, and discomfort. Demodetic mange, particularly in dogs, presents a difficult clinical problem for veterinarians, as it can involve the face and the entire body of the animal in some cases. Existing treatments can be expensive and are not always effective, with the result that affected animals are sometimes euthanized. Herein is proposed the use of Carvacrol to treat veterinary Demodex infections to provide comfort to the animal and prevent transmission to the humans.

In general, parasitic mites such as those causing mange are transmissible to humans.

Herein is described a composition of essential oils containing carvacrol and thymol to treat Demodex mites specifically as it related to blepharitis but also in areas which my seed the region of the eye lid such as the face. The unique ability to rapidly kill Demodex at a much lower concentration than other commercial essential oils products making it safe, tolerable and effective to be administered at home on a regular basis. As Demodex is ubiquitous on the skin and face of humans, having this product to be used as a proactive protection of Demodex mite overgrowth may be useful for some individuals. In addition, the product may be included for veterinary use to treat animals and prevent transmission to humans.

### Seborrheic Dermatitis

Seborrheic Dermatitis (SD) is a common inflammatory dermatosis most commonly encountered in adults, although infants and adolescents may be affected. The role of Malassezia spp in the pathogenesis of SD has long been suggested and debated in the literature. There are data to suggest that flares of SD are associated with an increase in the organism load of Malassezia spp, and improvement after therapy correlates with a reduction in the organism counts of these yeasts.

Malassezia furfur, also known as Pityrosporum ovale, is a yeast-like fungus that can contribute to blepharitis, especially in people with seborrheic dermatitis. Malassezia is a normal part of the skin's flora, but in some people, it can trigger seborrheic dermatitis, an inflammatory reaction.

Current therapies would include topical anti-fungal medications. Some describe cyclosporine which simply decreases the inflammatory response but does not eradicate the yeast.

Antifungal activities of the essential oils, Oregano vulgare and Thyme vulgaris, and their main component, carvacrol, were evaluated against 27 clinical isolates of Malassezia furfur. Minimum inhibitory concentrations (MICs) were measured according to the broth microdilution protocols by Clinical and Laboratory Standards Institute (CLSI) modified for Malassezia spp. EOs and carvacrol showed low MIC values ranged 450-900 μg/ml against M. furfur. No differences in EOs antifungal activity were observed in sensitive to resistant fluconazole isolates. The antifungal activity obtained showed O. vulgare EO, T. vulgaris EO and carvacrol, their compound, as potential antimicrobial agents against M. furfur, yeast associated with human mycoses.

The minimum inhibitory concentration (MIC) and minimum fungicidal concentration (MFC) values demonstrate the potent antimicrobial action of carvacrol across a wide range of pathogens.

Herein is described a composition of essential oils containing carvacrol and thymol to treat Seborrheic dermatitis specifically as it related to blepharitis but also in areas which may seed the region of the eye lid such as the face. In addition, the product may be provided for veterinary use to treat the animal and prevent transmission to humans.

### Anti-Inflammatory

Inflammation is a natural defense mechanism of the body designed to eliminate harmful stimuli, including exogenous pathogens, and initiate the healing process through various chemical mediators and signaling pathways. Since infections are often accompanied by massive inflammation, an important advantage of an antimicrobial agent would be its anti-inflammatory and antioxidant effects.

During normal cellular metabolism, free radicals or reactive oxygen intermediates are produced by cells. However, excessive accumulation of free radicals such as superoxide radicals, hydrogen peroxide, and hydroxyl radicals lead to tissue damage. Infectious diseases often develop inflammation because the controlled accumulation of inflammatory factors such as leukotrienes, prostaglandins, TNF-α, or interleukins (e.g., IL-1, IL-6, IL-8, IL-10) has a beneficial effect in the fight against microbes. For example, cytokines are the link between cell damage and inflammation symptoms (cell migration, swelling, fever, or hyperalgesia). They are produced and released by many cell types in response to inflammatory stimuli. It is worth emphasizing that the generally beneficial pro-inflammatory response, as a result of overstimulation, can contribute to the development of dangerous disease syndromes such as sepsis and septic shock. For this reason, it is essential that the antimicrobial drug utilized also has antioxidant and anti-inflammatory activity.

Serious infections are often accompanied by massive inflammation, which, in extreme cases, can lead to sepsis. Carvacrol prevents polyunsaturated fatty acid peroxidation by inducing SOD, GPx, GR, and CAT. It also contributes to the reduction in pro-inflammatory cytokines such as IL-6, IL-8, ENA-78, and GCP-2 in a dose-dependent manner. It also inhibits the synthesis of prostaglandins, affecting the cellular level of COX-2. It also reduces the level of the body's immune response generated by LPS. Carvacrol also affects heat shock proteins (Hsp), which are overexpressed in infection-induced inflammation

As noted carvacrol (5-isopropyl-2-methyl phenol, (FIG. 1) is a small monoterpene phenolic compound naturally found in the essential oils rich in plants of the Lamiaceae family including Oregano L., Thymus L., and Salvia L. Several studies have reported on the anti-inflammatory and analgesic properties of carvacrol using different infectious inflammatory cell/animal models. Furthermore, the potential mechanisms of carvacrol were reported as inhibiting the synthesis of TNF-α, PGE2, and nitric oxide and decreasing the levels of cytokines and COX-2. The anti-inflammatory benefits of a carvacrol solution can be increased by adding cannabidiol (CBD) oil to the essential oils forming the carvacrol mixture.

Carvacrol decreased the production of inflammatory cytokines, as well as matrix metalloprotease such as MMP-1, MMP-3, and MMP-13. Moreover, it also inhibited the activation pathways of TLR4/MyD88/NF-κB, p38, and ERK1/2, respectively.

It is worth emphasizing that in the concentrations utilized, the effect of carvacrol was comparable to that of dexamethasone—a glucocorticosteroid with strong and long-lasting anti-inflammatory, anti-allergic, and immunosuppressive effects.

Current anti-inflammatory therapies for the eyelids includes steroids. With long-term topical steroids can exacerbate infections as well as induce thinning of the eyelid skin and can even lead to blindness due to glaucoma or cataracts.

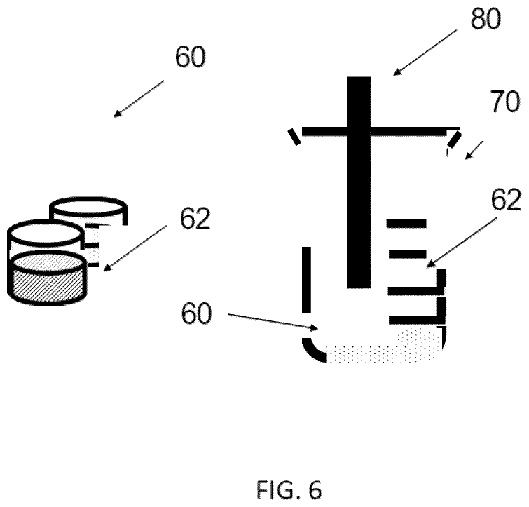

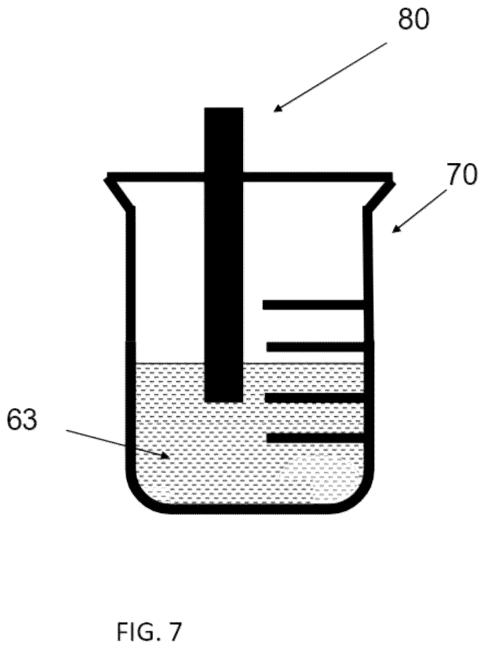

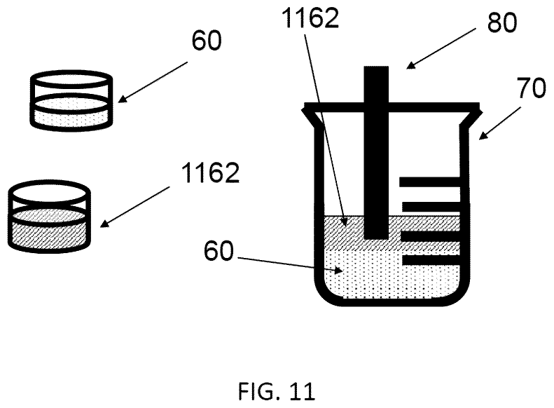

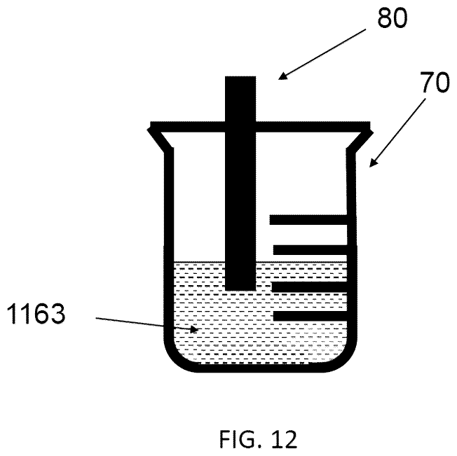

The formula includes a composition of essential oils containing carvacrol and thymol to treat inflammation specifically as it related to blepharitis but also in areas around of the eyelid such as the face. FIG. 6 illustrates the process of combining the carvacrol liquid 60 with oregano oil 62 in the glass beaker 70. The sonifier 80 mixes these components to create the carvacrol oil mixture 63, as shown in FIG. 7. An alternative formulation process is depicted in FIG. 11, where the carvacrol liquid 60 is combined with thymol oil 1162 in the glass beaker 70. This combination results in the liquid mixture 116 shown in FIG. 12 after sonication.

The carvacrol and oil mixture as shown in FIG. 6 is created by using any suitable high shear mixing technology or sonification device such as a Branson Sonifier 450 sonicating the carvacrol in the oregano oil. However, any suitable high shear mixing technology could be used such as a batch high-shear mixer, an inline high-shear rotor-stator mixer, an inline rotor-stator mixer or an ultra-high-shear inline mixer. An example of a typical preparation of the mixture is to take the 10 mg of carvacrol liquid 60 and place it in a 300-ml suitable glass container such as a beaker 70. Then add 200 ml of oregano oil 62, but any suitable oil described can be used. Then place the sonicator tip of a sonifier 80 so that it extends to approximately 2 mm from the bottom of the beaker. Set the power supply for controls accordingly to 20% Duty Cycle, output control to 5 and timer to 7. Cover the beaker with a piece of plastic film to prevent splatter or contamination and sonicate for 15 minutes until all the carvacrol liquid 60 is suspended in the oregano oil 62 forming carvacrol and oregano oil 63 as shown in FIG. 7. As described FIG. 6 illustrates a process for forming the composition. A carvacrol liquid 60 and an oregano oil 62 are combined in a glass beaker 70. A sonifier 80 extends into the glass beaker 70 to mix the components through sonication, creating a carvacrol oil mixture 63 as shown in FIG. 7.

The sonification can also be completed in other devices such as placing the glass container in an ultrasonic bath cleaner such as a Branson HT50 ultrasonic bath cleaner and sonicating the mixture until all the carvacrol liquid 60 is suspended in the oregano oil 62 forming carvacrol and oregano oil 63.

The sonication process typically lasts for a predetermined duration, often ranging from 5 to 15 minutes, depending on the specific formulation requirements. The power and frequency settings of the sonifier are adjusted based on the volume and viscosity of the liquids being mixed.

One should note that the container should not come in contact with the bottom of the ultrasonic bath cleaner. The formula may be used for veterinary use to treat the animals. The formulation can also be incorporated into an application medium such as a serum, salve, ointment, creams, tinctures, eye drop or eye wash to facilitate application to the Meibomian gland or other targeted body part.

The process can also use thyme oil. When using Thyme oil as the carrier one simply replaces the oregano oil 62 with Thyme oil and mix until the carvacrol liquid 60 is suspended in the Thyme oil. In an alternative embodiment the essential oil can include a CBD oil, a cannabis oil, and other cannabis-derived oils a CBD oil, a cannabis oil, and other cannabis-derived oils as a component of the

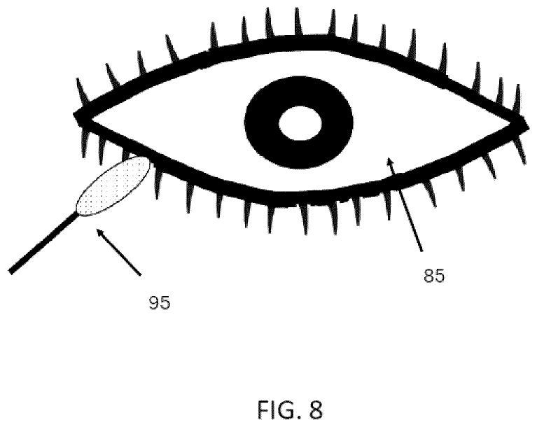

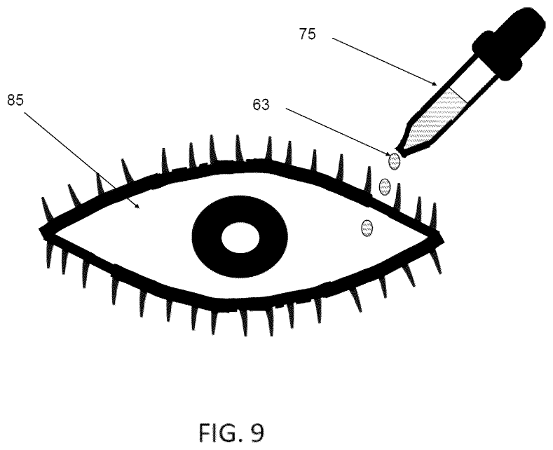

The carvacrol oil mixture 63 can be applied to an eye 85 using different methods. FIG. 8 depicts application using a swab 95. When using the swab 95 method, the user gently applies the carvacrol oil mixture to the eyelid margins, ensuring even coverage. The swab 95 is moved along the eyelid margins in a gentle, sweeping motion to distribute the treatment effectively. FIG. 9 shows application using a dropper tip 75 which enables controlled dispensing of the carvacrol oil mixture 63 onto the eye 85 and eyelid margins. For the dropper tip 75 and dropper vial 90 methods, the user tilts their head back slightly and carefully dispenses 1-2 drops of the carvacrol oil mixture 63 onto the eyelid margins or directly onto the eye 85. The user then gently closes their eyes to allow the treatment to spread evenly across the affected areas.

The composition exhibits strong antimicrobial properties, contributing to its effectiveness in treating various eyelid conditions. The broad-spectrum antimicrobial activity of the composition, combined with its effectiveness against Demodex mites, makes it a comprehensive treatment for various eyelid conditions. The composition addresses multiple potential causes of eyelid issues, including bacterial and fungal infections, as well as Demodex infestations. This multi-faceted approach to treating eyelid conditions sets the composition apart from traditional single-target treatments.

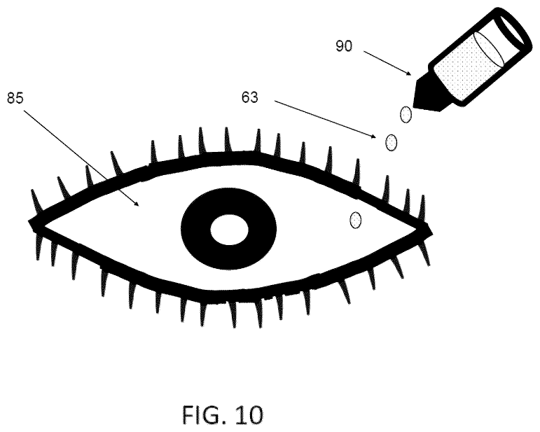

The application methods depicted in FIG. 8, FIG. 9, and FIG. 10 allow for precise delivery of the antimicrobial composition to the affected eyelid areas. The swab application method shown in FIG. 8 enables direct contact of the carvacrol oil mixture with the eyelid margins, ensuring effective treatment of microbial infections. The dropper tip and dropper vial methods illustrated in FIG. 9 and FIG. 10, respectively, provide controlled administration of the antimicrobial composition to the eye and eyelid margins.

These application methods provide flexibility in administering the carvacrol oil mixture, allowing users to choose the most suitable technique for their specific needs and preferences. The precise application ensures that the treatment reaches the targeted areas effectively, maximizing the therapeutic benefits of the carvacrol oil mixture for various eyelid conditions.

The versatility of the carvacrol oil mixture is enhanced by its various application methods. The swab application illustrated in FIG. 8 allows for targeted treatment of eyelid margins. The dropper tip method shown in FIG. 9 and the dropper vial method in FIG. 10 enable precise administration to the ocular surface. These diverse application options facilitate patient compliance and ensure effective delivery of the active components.

FIG. 10 illustrates application using a dropper vial 90. The preparation of the composition involves combining carvacrol liquid with a therapeutic oil using sonication. FIG. 6 illustrates the process of forming the carvacrol oil mixture. The carvacrol liquid 60 and oregano oil 62 are placed in the glass beaker 70. The sonifier 80 extends into the glass beaker 70 to mix the components.

The preparation of the composition involves combining carvacrol liquid with a therapeutic oil using sonication. FIG. 6 illustrates the process of forming the carvacrol oil mixture. The carvacrol liquid 60 and oregano oil 62 are placed in the glass beaker 70. The sonifier 80 extends into the glass beaker 70 to mix the components.

The sonication process uses high-frequency sound waves to agitate and combine the carvacrol liquid with the therapeutic oil. The sonifier 80 generates these sound waves, causing cavitation bubbles to form and collapse rapidly. This action creates intense local heating and high-pressure regions, effectively dispersing the carvacrol liquid throughout the therapeutic oil.

An alternative formulation process is shown in FIG. 11, where a carvacrol liquid 60 is combined with a thymol oil 1162 in a glass beaker 70. FIG. 12 depicts the resulting liquid mixture 116 in the glass beaker 70 after sonication.

Referring now to FIG. 7 there is shown sonifier 80 and the carvacrol and oregano oil 63 in beaker 70 after sonication.

The carvacrol and oregano oil 63 can be placed on a swab 95 and the swab 95 can be used to apply the carvacrol and oregano oil 63 to the eyelid margins 86 or eye 85 as shown in FIG. 8.

The carvacrol and oregano oil 63 can be placed in an eye dropper 75 and the drops applied to the eye 85 and eyelid margins 85 as shown in FIG. 9.

Alternatively, the carvacrol and oregano oil 63 can be placed in an eye dropper vial 90 and the drops applied to the eye 85 and eyelid margins 85 as shown in FIG. 10.

After sonication, the carvacrol oil mixture is ready for application. FIG. 8 demonstrates application using the swab 95 to treat the eye 85. FIG. 9 shows the use of the dropper tip 75 for administering the carvacrol oil mixture 63 to the eye 85. FIG. 10 illustrates the application using the dropper vial 90 to dispense the carvacrol oil mixture 63 onto the eye 85.

The preparation method ensures thorough mixing of the carvacrol with the therapeutic oil, creating a uniform composition for effective treatment of eyelid conditions. The carvacrol oil mixture when applied to the eyelid margins using various methods to effectively treat eyelid conditions. FIG. 8 illustrates the application of the carvacrol oil mixture using a swab 95. The swab 95 is positioned near the eye 85 and used to apply the carvacrol oil mixture directly to the eyelid margins. This method allows for precise application and ensures the treatment reaches the affected areas.

One should be aware that sterilizing containers use to store the oils is crucial to prevent bacterial growth which can lead to infections.

The carvacrol oil mixture is formulated as a cream, serum, salve, or lotion. The cream formulation of the carvacrol oil mixture is created by combining the carvacrol oil mixture 63 with a wax and water. The components are heated in a suitable container and whisked thoroughly to create a stable emulsion. The serum formulation of the carvacrol oil mixture is prepared by combining the carvacrol oil mixture 63 with other lightweight oils and emulsifiers to create a thin, easily absorbed product. The salve formulation of the carvacrol oil mixture is made by gently melting a wax in a heatproof bowl over simmering water, then adding the carvacrol oil mixture 63 and stirring until thoroughly combined. The lotion formulation of the carvacrol oil mixture is created by combining the carvacrol oil mixture 63 with water and emulsifiers to create a light, hydrating product.

There are three primary types of topical treatments envisioned: infused oils, serum, salves, and creams or lotions.

The first is an infused oils are made by imbibing active ingredients in a carrier oil like olive or almond to extract the medicinal plant constituents that will help soothe your skin. The infused oils can be made using a couple of different methods:
a. Cold Infusion: This method is best suited for delicate herbs. It involves combining carvacrol and oregano oil 63 in a sealed jar and letting it infuse in a cool, dark place for 2-6 weeks, shaking periodically. Afterward, the oil is strained and stored.
b. Warm Infusion: This is a faster method as the carvacrol and oregano oil 63 is heated gently in an appropriate container for a short time, then cooled, strained, and stored.

The second is a salve. Salves are solid combinations of active ingredients combined with oils and a wax such as beeswax or soy wax.
a. The salve is made by gently melting the wax such as a beeswax in a heatproof bowl over a pot of simmering water.
b. Combine carvacrol and oregano oil 63 and wax: Add the carvacrol and oregano oil 63 to the melted wax and stir until thoroughly combined.
c. Pour the warm salve mixture into clean containers.

Creams, serums and lotions vary from thicker to more light in texture, and combine active ingredients, oils with water to create a hydrating topical treatment.

A carvacrol and oregano oil 63 cream is made by combining the carvacrol and oregano oil 63 with other a wax such as beeswax and water. Then combining it with other cream ingredients to create a stable emulsion.
a. Using a suitable container, you heat the carvacrol and oregano oil 63 and a suitable wax such as beeswax (for thickening), and water.
b. Whisk the mixture thoroughly to create a stable emulsion where the oil and water are well combined.

When formulating the application medium, the amount of carvacrol liquid needs to be controlled to control the concentration level of the treatment. A final concentration by weight of carvacrol liquid to the application medium is from 1 to 80 percent by weight of carvacrol to application medium weight.

### Formulation

Carvacrol and Thymol is a component of essential oils obtained from many plants, e.g., Corido thymus, Lippia pepperwort, black cumin (Nigella sativa), oregano (Origanum compactum), O. dictamnus, O. microphyllum, O. onites, O. scabrum, O. vulgare, pepperwort (Lepidium flavum), wild bergamot (Citrus aurantium var. bergamia Loisel), Monarda didyma, thyme (Thymus glandulosus), cannabidiol (CBD) oil and savory (Satureja hortensis).

In some cases, an alternative formulation process is used, as depicted in FIG. 11. The carvacrol liquid 60 is combined with thymol oil 1162 in the glass beaker 70. FIG. 12 illustrates the liquid mixture 1116 in the glass beaker 70 following sonication. The resulting mixture can have a carvacrol liquid 60 component of 0.5 to 80% depending on the nature of the desired mixture of carvacrol liquid 60 and the selected carrier or essential oil used.

One embodiment of the treatment includes formulations for carvacrol and thymol oil. The formulation of the carvacrol and thymol oil is achieved by the similar process as used with the carvacrol and thymol oil 1162. First the carvacrol and oil mixture as shown in FIG. 11 and FIG. 12 is created by using any suitable high shear mixing technology or sonification device such as a Branson Sonifier 450 sonicating the carvacrol in the thymol oil. However, any suitable high shear mixing technology could be used such as a batch high-shear mixer, an inline high-shear rotor-stator mixer, an inline rotor-stator mixer or an ultra-high-shear inline mixer. An example of a typical preparation of the mixture is to take the 10 mg of carvacrol liquid 60 and place it in a 300-ml suitable glass container such as a beaker 70. Then add 200 ml of thymol oil 1162, but any suitable oil described can be used. Then place the sonicator tip of a sonifier 80 so that it extends to approximately 2 mm from the bottom of the beaker. Set the power supply for controls to approximately 20% Duty Cycle, output control to 5 and timer to 7. Cover the beaker with a piece of plastic film to prevent splatter or contamination and sonicate for 15 minutes until all the carvacrol liquid 60 is suspended in the thymol oil 1162 forming carvacrol and thymol oil 1163 as shown in FIG. 12. The resulting mixture can have a carvacrol liquid 60 component of 0.5 to 80% depending on the nature of the desired mixture of carvacrol liquid 60 and the selected carrier or essential oil used.

The topical application is the most common method of administration of ophthalmic medicaments. The topical route makes possible the instillation into the eye of drops or the application in the eye of solutions, eyewashes, suspensions, salves, serums, ointments, gels, sprays, foams, powders, lotions, viscoelastic solutions and/or the deploying of solid forms at the surface of the eye, impregnated pads, syndets or wipes.

Some formulations can also be provided in the form of suspensions of microspheres or nanospheres or of vesicles formed from lipid or polymer or of polymeric patches and of hydrogels making possible controlled release. These compositions for topical application can be provided in anhydrous form, in aqueous form or in the form of an emulsion.

The pharmaceutical compositions for topical application are preferably non-irritating and compatible with the tissues of the eye. The solutions can be sterile preparations and can be free from all particles. The suspensions can be sterile preparations and can include solid particles in a liquid vehicle appropriate for ocular instillation. The ointments can be semisolid and sterile preparations.

Orally, the pharmaceutical compositions can be provided in liquid, pasty or solid form, in the form of powders and more particularly in the form of tablets, including sugar-coated tablets, hard gelatin capsules, syrups, suspensions, solutions, powders, granules, emulsions, microspheres or nanospheres or vesicles formed from lipid or polymer making possible controlled release. Parenterally, the compositions can be provided in the form of solutions or suspensions for infusion or for injection. Rectally, the compositions can be provided in the form of suppositories. In some cases, the pharmaceutical compositions are topical ophthalmic compositions, and not oral or rectal compositions.

In some embodiments, the compositions according to the invention are provided in the form of an eyewash or of eye drops. The term “eyewash” means a liquid formulation specifically appropriate for administration to the conjunctiva of the eye and the cornea. The eyewash can include a volume of the instilled drops of, e.g., approximately 25-50 microliters.

The compositions can in some embodiments comprise from 0.001% to 100% of at least one compound as disclosed herein, by weight with respect to the total weight of the composition.

Carvacrol (and Thymol) is a component of essential oils obtained from many plants, e.g., Corido thymus, Lippia pepperwort, black cumin (Nigella sativa), oregano (Origanum compactum), O. dictamnus, O. microphyllum, O. onites, O. scabrum, O. vulgare, pepperwort (Lepidium flavum), wild bergamot (Citrus aurantium var. bergamia Loisel), Monarda didyma, thyme (Thymus glandulosus), and savory (Satureja hortensis).

A contact lens impregnated or treated with a carvacrol solution may be used to prevent ocular infections and or treat existing ones. The incorporation of the carvacrol oil mixture into contact lenses offers several advantages. First, it ensures consistent delivery of the therapeutic components directly to the ocular surface. Second, it combines vision correction with treatment, improving patient compliance. Third, it provides a barrier against potential pathogens, reducing the risk of contact lens-associated infections.

For the synthesis of carvacrol-delivering nanoparticles by ionotropic gelation, using chitosan due to its biocompatibility, biodegradability, intrinsic antibacterial activity, and hemostatic and mucoadhesive properties.

In certain embodiments the nanoparticles of carvacrol can be added to an antioxidant solution such as 2% N-acetyl-carnosine (NAC), astaxanthin, resveratrol, quercetin, lutein, zeaxanthin, Riboflavin, catalase, glutathione, vitamin E and vitamin C (ascorbic acid).

In some embodiments, methods may be used to obtain carvacrol which is included in the formulation. carvacrol, or cymophenol, C6H3(CH3)(OH)C3H7, is a monoterpenoid phenol. It has a characteristic pungent, warm odor of oregano. Carvacrol is present in the essential oil of Origanum vulgare (oregano), oil of thyme, oil obtained from pepperwort, and wild bergamot. The essential oil of thyme subspecies contains between 5% and 80% of carvacrol, while Satureja (savory) subspecies have a content between 1% and 45%. Origanum majorana (marjoram) and Dittany of Crete are rich in carvacrol, 50% and 60-80% respectively. In vitro, carvacrol has antimicrobial activity against 25 different phytopathogenic bacteria and strains including: Cladosporium herbarum, Penicillium glabrum, Pseudomonas syringae, and fungi such as Fusarium verticillioides/F. moniliforme, Rhizoctonia solani/R. solani, Sclerotinia sclerotiorum, and Phytophthora capsici. Carvacrol may be synthetically prepared by a number of routes. The fusion of cymol sulfonic acid with caustic potash results in desulfonation. By the action of nitrous acid on 1-methyl-2-amino-4-propyl benzene, one effects diazotization. Prolonged heating of camphor and iodine or carvone with glacial phosphoric acid have also been demonstrated. The dehydrogenation of carvone with a palladium-carbon catalyst has been established. It has also been prepared by transalkylation of isopropylated cresols. It is extracted from Origanum oil by means of a 50% potash solution. It is a thick oil that sets at −20° C. to a mass of crystals of melting point 0° C., and boiling point 236-237° C. Oxidation with ferric chloride converts it into dicarvacrol, whilst phosphorus pentachloride transforms it into chlorcymol.

There are multiple causes for blepharitis that can lead to a number of ocular diseases. Diagnosing the exact ideology of the blepharitis is often difficult. Various individual therapies are used for each of the different etiologies, but they are fraught with both short term and long-term disadvantages. Not to mention the need for an expensive multi-drug approach often initiated only after the blepharitis has become severe. Broad spectrum treatment and prophylaxis of this far-reaching disease is possible. Much like the long-standing dental model used to prevent tooth decay and gum disease. Having one product that is available to simultaneously treat multiple forms of blepharitis on a regular basis at an affordable price would offer the public a way to treat and prevent this debilitating disease process. This composition of essential oils containing carvacrol and thymol to treat blepharitis in its various forms fills an unmet need.

Cannabidiol (CBD) and cannabinoids extracted from hemp plants (Cannabis sativa, Cannabis indica, Cannabis ruderalis) helps relax muscles, reduce spasticity, rigidity, and tension as a result of stress or overwork. Specifically, Cannabidiol is a chemical in the Cannabis sativa plant, also known as marijuana or hemp. There are over 80 chemicals, known as cannabinoids, which have been identified in the Cannabis sativa plant. The preferred form is as a high percentage CBD distillate (85-95% CBD), which also contains other components distilled from the plant, which includes but is not limited to secondary components such as terpenes, flavonoids, other cannabinoids and compounds. The cannabidiol along with these secondary components, contribute to what is termed the entourage effect. The entourage effect is a mechanism by which cannabis compounds other than tetrahydrocannabinol act synergistically to modulate the overall psychoactive effects of the cannabidiol distillate. The process used to produce the high cannabidiol content wax distillate contains zero or undetectable levels of the psychoactive component Delta-9 THC. Based on the particular cannabidiol percentage level in a sample of wax distillate, the formulation would need to be adjusted to achieve the determined amount of cannabidiol.

A composition includes carvacrol and a therapeutic oil. The therapeutic oil may be selected from a group that includes, but is not limited to, an essential oil, a CBD oil, a cannabis oil, or other cannabis-derived oil, or any combination thereof. The carvacrol and the therapeutic oil may be combined to form a mixture. In some cases, the combination of carvacrol and the therapeutic oil may be achieved through a process such as sonication. The composition may be particularly useful for treating eyelid conditions such as blepharitis. The use of carvacrol and therapeutic oil in the composition may provide certain benefits, such as potentially offering a natural and effective treatment for eyelid conditions.

In addition to carvacrol, the composition may also comprise a therapeutic oil. The therapeutic oil may be selected based on its potential therapeutic benefits. For instance, an essential oil may be included in the composition. In some cases, the therapeutic oil may be a CBD oil or a cannabis oil. These oils, derived from the cannabis plant, may offer potential anti-inflammatory and analgesic properties. Other cannabis-derived oils may also be used in the composition.

In some aspects, the carvacrol and the therapeutic oil may be combined to form a mixture. The process of combining these components may vary. For example, the carvacrol may be sonicated in the therapeutic oil. This process may facilitate the dispersion of carvacrol in the oil, potentially enhancing the effectiveness of the composition.

It should be noted that the composition may be formulated in various forms suitable for application to the eyelid. These forms may include, but are not limited to, creams, serums, gels, ointments, or lotions. The specific form of the composition may be selected based on factors such as the specific eyelid condition to be treated, the desired duration of treatment, and the patient's preferences.

Carvacrol, a key component of the composition, is a monoterpenoid phenol that may be derived from various plant sources. In some cases, carvacrol may be obtained from oregano, thyme, or other plants known to contain this compound. Carvacrol may be characterized by its pungent, warm odor, which is often associated with oregano. In terms of its chemical structure, carvacrol may be represented by the formula C10H14O and may possess a hydroxyl group (—OH) attached to a cyclic hydrocarbon ring, which may contribute to its phenolic nature.

Carvacrol may exhibit a range of therapeutic properties that may be beneficial in the context of treating eyelid conditions. For instance, carvacrol may possess antimicrobial properties, which may be useful in addressing eyelid conditions associated with bacterial or fungal infections. In some cases, carvacrol may exhibit antifungal activity against a variety of fungal species, potentially making it a suitable component for treating eyelid conditions caused by fungal infections.

In addition to its antimicrobial properties, carvacrol may also exhibit anti-inflammatory properties. Inflammation may be a common symptom in many eyelid conditions, and the potential anti-inflammatory effects of carvacrol may contribute to the alleviation of such symptoms.

Furthermore, carvacrol may also exhibit analgesic properties, which may be beneficial in providing relief from pain or discomfort associated with certain eyelid conditions. In some cases, the analgesic properties of carvacrol may be attributed to its potential ability to inhibit the synthesis of certain pain-inducing substances in the body.

It should be noted that the concentration of carvacrol in the composition may vary. In some cases, the concentration of carvacrol may be selected based on factors such as the specific eyelid condition to be treated, the severity of the condition, and the desired therapeutic effect. The optimal concentration of carvacrol may be determined through routine experimentation by those skilled in the art.

In certain embodiments, the process of combining carvacrol and the therapeutic oil to form a mixture involves the use of sonication. Sonication, a technique that uses sound waves to agitate particles in a solution, may be employed to facilitate the dispersion of carvacrol in the therapeutic oil. This process may involve placing the carvacrol and the therapeutic oil in a sonication device, which may then generate sound waves that cause the carvacrol to disperse evenly throughout the oil. The duration and intensity of sonication may vary, and may be selected based on factors such as the specific properties of the carvacrol and the therapeutic oil, and the desired characteristics of the resulting mixture.

In certain embodiments, the sonication process can be performed at room temperature. However, in other cases, the sonication process may be performed at an elevated temperature. The specific temperature at which the sonication process is performed may depend on factors such as the specific properties of the carvacrol and the therapeutic oil, and the desired characteristics of the resulting mixture.

In certain embodiments, the sonication process can be performed in a controlled environment, such as a laboratory or a manufacturing facility. The sonication device used in the process may be a standard sonication device known to those skilled in the art. The specific settings of the sonication device, such as the frequency and amplitude of the sound waves, may be selected based on factors such as the specific properties of the carvacrol and the therapeutic oil, and the desired characteristics of the resulting mixture.

In certain embodiments, the sonication process can result in a mixture of carvacrol and the therapeutic oil that is homogeneous, meaning that the carvacrol is evenly dispersed throughout the oil. In other cases, the sonication process may result in a mixture that is not completely homogeneous, but still suitable for use in treating eyelid conditions. The specific characteristics of the resulting mixture may depend on factors such as the specific properties of the carvacrol and the therapeutic oil, the sonication process parameters, and the desired therapeutic effect. It should be noted that other methods of combining carvacrol and the therapeutic oil may also be used. For instance, in some cases, the carvacrol and the therapeutic oil may be combined using a stirring or shaking process. In other cases, the carvacrol and the therapeutic oil may be combined using a heating process. The specific method of combining the carvacrol and the therapeutic oil may be selected based on factors such as the specific properties of the carvacrol and the therapeutic oil, and the desired characteristics of the resulting mixture.

In certain embodiments, the composition can include varying ratios or concentrations of carvacrol and the therapeutic oil. The specific ratio or concentration of carvacrol and the therapeutic oil may be selected based on factors such as the specific eyelid condition to be treated, the severity of the condition, the desired therapeutic effect, and the specific properties of the carvacrol and the therapeutic oil.

For instance, in some cases, the composition can include a higher concentration of carvacrol relative to the therapeutic oil. This may be suitable for treating more severe eyelid conditions, or for achieving a stronger therapeutic effect. In other cases, the composition may include a lower concentration of carvacrol relative to the therapeutic oil. This may be suitable for treating milder eyelid conditions, or for achieving a gentler therapeutic effect.

The composition can include equal concentrations of carvacrol and the therapeutic oil. This may provide a balanced therapeutic effect, potentially suitable for treating a wide range of eyelid conditions.

The composition can include a concentration of carvacrol that is within a range of about 0.1% to about 10% by weight of the composition. In other cases, the concentration of carvacrol may be within a range of about 1% to about 5% by weight of the composition and in other cases the concentration of carvacrol may be within a range of about 0.5% to about 80% by weight. However in a preferred embodiment the concentration of carvacrol may be within a range of about 1% to about 5% by weight of the composition. The specific concentration of carvacrol may be selected based on routine experimentation by those skilled in the art. Similarly, the concentration of the therapeutic oil in the composition may also vary. In some cases, the concentration of the therapeutic oil may be within a range of about 0.1% to about 100% by weight of the composition. In other cases, the concentration of the therapeutic oil may be within a range of about 1% to about 100% by weight of the composition. The specific concentration of the therapeutic oil may be selected based on routine experimentation by those skilled in the art. It should be noted that the specific ratio or concentration of carvacrol and the therapeutic oil in the composition may be adjusted as needed to achieve the desired therapeutic effect. The optimal ratio or concentration of carvacrol and the therapeutic oil may be determined through routine experimentation by those skilled in the art.

The composition can be used to treat a variety of eyelid conditions. While blepharitis has been mentioned as one such condition, it should be noted that the composition may also be effective in treating other eyelid conditions. For instance, the composition may be used to treat conditions such as chalazion, a condition characterized by the formation of a lump or cyst in the eyelid due to a blocked oil gland. The anti-inflammatory properties of the composition may potentially help reduce the swelling associated with this condition.

The composition can be used to treat styes, which are painful, red bumps that form at the base of an eyelash or under the eyelid. The antimicrobial properties of the composition may potentially help combat the bacterial infection causing the stye. The composition may be used to treat conditions such as dry eye syndrome, a condition characterized by insufficient tear production or poor tear quality. The composition may potentially help soothe the dryness and irritation associated with this condition.

The composition can be used to treat conditions such as eyelid dermatitis, a condition characterized by inflammation of the skin on or around the eyelids. The anti-inflammatory and analgesic properties of the composition may potentially help alleviate the symptoms of this condition. The composition may be used to treat conditions such as meibomian gland dysfunction, a condition characterized by the abnormal functioning of the meibomian glands in the eyelids, which are responsible for producing the oily part of tears. The composition may potentially help improve the quality of the oil produced by these glands, thereby alleviating the symptoms of this condition. It should be noted that the specific eyelid condition to be treated may influence the formulation of the composition. For instance, the concentration of carvacrol and the therapeutic oil in the composition may be adjusted based on the specific eyelid condition to be treated. The optimal formulation of the composition for treating a specific eyelid condition may be determined through routine experimentation by those skilled in the art.

The use of the composition for treating eyelid conditions may offer potential benefits over existing treatments. For instance, the composition may provide a natural alternative to synthetic medications, which may be preferred by some individuals due to concerns about potential side effects or interactions with other medications. The composition may also offer a more targeted approach to treatment, potentially addressing the specific symptoms of the eyelid condition more effectively than broad-spectrum treatments.

The composition may provide a more convenient treatment option. For example, the composition may be formulated as a topical application, which may be easier to administer than oral medications or injections. This may be particularly beneficial for individuals who have difficulty swallowing pills or who are uncomfortable with injections. The composition may offer a more cost-effective treatment option. The components of the composition, such as carvacrol and the therapeutic oil, may be derived from readily available plant sources, potentially reducing the cost of production. This may result in a lower cost for the end user, making the treatment more accessible to a wider range of individuals.

In some aspects, the composition may provide a more versatile treatment option. The composition may be effective in treating a variety of eyelid conditions, potentially reducing the need for multiple medications. This may be particularly beneficial for individuals with multiple eyelid conditions, or for those who experience different conditions at different times.

In other cases, the composition may offer potential benefits in terms of patient compliance. The composition may be more pleasant to use than other treatments, potentially encouraging individuals to adhere to their treatment regimen. For instance, the composition may have a pleasant aroma due to the inclusion of essential oils, which may enhance the user's experience and encourage regular use.

In some aspects, the composition may offer potential benefits in terms of safety. The composition may have a lower risk of side effects or adverse reactions compared to some existing treatments, potentially making it a safer option for individuals with sensitive skin or those with allergies to certain medications.

It should be noted that the potential benefits of using the composition for treating eyelid conditions may vary depending on factors such as the specific eyelid condition to be treated, the severity of the condition, the individual's overall health status, and the individual's preferences and needs.

Certain embodiments can be formulated to address user feed back such as sting sensation and odor of the mixture while still having potential affect against the periorbital organisms. The mixtures can have a range of carvacrol and the applicant tested multiple concentrations of carvacrol until a range of carvacrol concentrations was found to be effective and also provided a comfortable experience for a majority of patients. In addition, the applicant found that adding a fragrance enhanced the patient experience. The inclusion of various essential oils provided positive patient experience. The preferred mixture included carvacrol, lavender, peppermint and bergamot. The lavender, peppermint and bergamot mixture provides a fragrance that was preferred by a majority of patients.

The applicant conducted a series of test to validate the mixture and process of formulating the mixture with patients that had a high concentration of Demodex mites. The patients were asked to use the liquid product for two weeks. First the patients had a baseline dry eye test done to provide a reference and the patients filled out as Ocular Surface Disease Index (OSDI) survey of dry eye symptoms. The score pretreatment score was recorded and the patients took a second OSDI survey after the study and the majority of the patients showed improvement in patent symptoms after 2 weeks of treatment. The average pretreatment Tear Break Up Time (TBUT) was 6 seconds which improved to 17 seconds suggesting a decrease in rate of tear evaporation. Lastly, the average number of Meibomian glands yielding liquid secretion on a lower eyelid increased after 2 weeks of treatment suggesting better oil produced for stability of the tears. The results of the testing of the carvacrol, lavender, peppermint and bergamot mixture resulted in a 100% of the patients approving of the treatment and fragrance of the product. Also, 100% of the patients wanted to continue to use the product.

In addition, the formulation can be adjusted to incorporate fragrance enhancers including various essential oils, to provide a fragrance acceptable to most users. The embodiment preferred by the patients was a liquid formulation comprised of 0.3% carvacrol and 1% Lavender/Bergamot/Peppermint Blend.

The formulations tested included:
0.3% Carvacrol and 1% Lavender/Bergamot/Peppermint Blend.
a. Water (Aqua)
b. Allantoin
c. Aloe Barbadensis Leaf Juice
d. Caffeine
e. Sodium Hyaluronate
f. Carbomer
g. Propanediol
h. Xanthan Gum
0.3% Carvacrol with Water (Aqua), Tetrasodium Glutamate Diacetate, Sodium Hydroxide
0.3% Carvacrol with Leuconostoc/Radish Root Ferment Filtrate, Water (Aqua), Lactobacillus, Cocos Nucifera (Coconut) Fruit Extract
0.3% Carvacrol with Magnesium Chloride, Tetradecyl Aminobutyroylvalylaminobutyric Urea Trifluoroacetate, Glycerin
0.3% Carvacrol with Panthenol, Water (Aqua), Pantolactone
0.3% Carvacrol with Hydroxypropyl Cyclodextrin, Palmitoyl Tripeptide-38, Glycerin, Water (Aqua)
0.3% Carvacrol with Japonica (Honeysuckle) Flower Extract, Lonicera Caprifolium (Honeysuckle) Flower Extract, Water (Aqua)
0.3% Carvacrol with Sorbitan Oleate Decylglucoside Crosspolymer, Water (Aqua), Sodium Chloride
a. Mentha Piperita Oil
b. Citrus Aurantium Bergamia (Bergamot) Fruit Oil
c. Lavandula Angustifolia Oil
d. Carvacrol

Any of the mixtures can be have Lavender/Bergamot/Peppermint Blend incorporated into the mixture.

The mixture had 0.3 carvacrol determined by weight of the final mixture, Water (Aqua), Allantoin, Aloe Barbadensis, Leaf Juice, Caffeine, Sodium Hyaluronate, Carbomer, Propanediol, Xanthan Gum. However, the carvacrol mixture tested that provided acceptable results was 0.05-5% by weight. The range of fragrance that provided the best patient results was 0.025-6% by weight.

The next preferred embodiment by the patients was a liquid formulation comprised 0.3% carvacrol and 1% mixture of Water (Aqua), Tetrasodium Glutamate Diacetate, Sodium Hydroxide. However, the carvacrol mixture tested that provided acceptable results was 0.05-4.5% by weight. The range of fragrance that provided the best patient results was 0.025-7% by weight.

The next preferred embodiment by the patients was a liquid formulation comprised 0.3% carvacrol and 1% mixture of Leuconostoc/Radish Root Ferment Filtrate, Water (Aqua), Lactobacillus, Cocos Nucifera and (Coconut) Fruit Extract. However, the carvacrol mixture tested that provided acceptable results was 0.02-4% by weight. The range of fragrance that provided the best patient results was 0.05-7% by weight.

The next preferred embodiment by the patients was a liquid formulation comprised 0.3% carvacrol and 1% mixture of Magnesium Chloride, Tetradecyl Aminobutyroylvalylaminobutyric Urea Trifluoroacetate and Glycerin. However, the carvacrol mixture tested that provided acceptable results was 0.01-45% by weight. The range of fragrance that provided the best patient results was 0.05-5% by weight.

The next preferred embodiment by the patients was a liquid formulation comprised 0.3% carvacrol and 1% mixture of Panthenol, Water (Aqua), Pantolactone. However, the carvacrol mixture tested that provided acceptable results was 0.01-4.5% by weight. The range of fragrance that provided the best patient results was 0.05-5% by weight.

The next preferred embodiment by the patients was a liquid formulation comprised 0.3% carvacrol and 1% mixture of Hydroxypropyl Cyclodextrin, Palmitoyl Tripeptide-38, Glycerin, Water (Aqua). However, the carvacrol mixture tested that provided acceptable results was 0.01-5% by weight. The range of fragrance that provided the best patient results was 0.05-5% by weight.

The next preferred embodiment by the patients was a liquid formulation comprised 0.3% carvacrol and 1% mixture of Japonica (Honeysuckle) Flower Extract, Lonicera Caprifolium (Honeysuckle) Flower Extract, Water (Aqua). However, the carvacrol mixture tested that provided acceptable results was 0.01-4.5% by weight. The range of fragrance that provided the best patient results was 0.05-5% by weight.

The next preferred embodiment by the patients was a liquid formulation comprised 0.3% carvacrol and 1% mixture of Sorbitan Oleate Decylglucoside Crosspolymer, Water (Aqua), Sodium Chloride. However, the carvacrol mixture tested that provided acceptable results was 0.05-5% by weight. The range of fragrance that provided the best patient results was 0.05-5% by weight.

The next preferred embodiment by the patients was a liquid formulation comprised 0.3% carvacrol and 1% mixture of Mentha Piperita Oil, Citrus Aurantium, Bergamia (Bergamot) Fruit Oil. Lavandula Angustifolia Oil and Carvacrol. However, the carvacrol mixture tested that provided acceptable results of the total carvacrol mixture was 0.1-6% by weight. The range of fragrance that provided the best patient results was 0.05-5% by weight.

The innovation can also be described as A composition for treating eyelid conditions, comprising:
carvacrol; and
   a therapeutic oil selected from the group consisting of an essential oil, a CBD oil, a cannabis oil, and other cannabis-derived oils,
   wherein the carvacrol and the therapeutic oil are combined to form a mixture.

The carvacrol composition wherein the carvacrol is sonicated in the therapeutic oil.

The carvacrol composition wherein the composition is in a form selected from the group consisting of a cream, a serum, a gel, an ointment, and a lotion.

The carvacrol composition wherein the composition is formulated for topical application to an eyelid.

The carvacrol composition wherein the composition is effective for treating blepharitis.

The carvacrol composition wherein the composition is effective for treating at least one of: chalazion, stye, dry eye syndrome, eyelid dermatitis, and meibomian gland dysfunction.

The carvacrol composition wherein the carvacrol is present in a concentration range of 0.1% to 10% by weight of the composition.

A method of treating an eyelid condition, comprising:
applying a composition to an eyelid of a subject, the composition comprising carvacrol and a therapeutic oil selected from the group consisting of an essential oil, a CBD oil, a cannabis oil, and other cannabis-derived oils,
wherein the carvacrol and the therapeutic oil are combined to form a mixture.

The method of using the carvacrol composition wherein the eyelid condition is blepharitis.

The method of using the carvacrol composition wherein the eyelid condition further includes at least one of: chalazion, stye, dry eye syndrome, eyelid dermatitis, and meibomian gland dysfunction.

The method of using the carvacrol composition wherein the composition is applied topically to the eyelid.

The method of using the carvacrol composition, a serum, a gel, an ointment, and a lotion.

The method of using the carvacrol composition wherein the carvacrol is present in a concentration range of 0.1% to 10% by weight of the composition.

The method of using the carvacrol composition, wherein the therapeutic oil is present in a concentration range of 0.1% to 100% by weight of the composition.

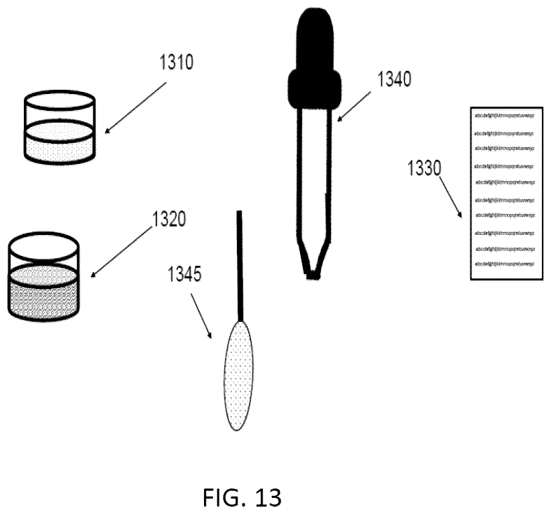

As shown in FIG. 13 a kit for treating an eyelid condition, comprising: a composition comprising carvacrol and a therapeutic oil selected from the group consisting of an essential oil, a CBD oil, a cannabis oil, and other cannabis-derived oils, wherein the carvacrol and the therapeutic oil are combined to form a mixture; and instructions for use.

The kit wherein the composition is in a form selected from the group consisting of a cream, a serum, a gel, an ointment, and a lotion.

The kit wherein the composition is effective for treating blepharitis.

The kit wherein the composition is effective for treating at least one of: chalazion, stye, dry eye syndrome, eyelid dermatitis, and meibomian gland dysfunction.

The kit wherein the carvacrol is present in a concentration range of 0.1% to 10% by weight of the composition.

The kit wherein the therapeutic oil is present in a concentration range of 0.1% to 100% by weight of the composition.

Referring to FIG. 1-FIG. 13 and preferably FIG. 1, illustrates a table displaying antimicrobial activity data for various microorganisms tested against different determination methods. The table includes three columns labeled “Microorganism,” “Determination Method,” and “MIC and/or MFC Value.” The table lists multiple microorganisms including Aspergillus flavus, various Candida species, Penicillium, Saccharomyces cerevisiae, and several bacterial strains. For each microorganism, the table specifies the determination method used, such as serial dilution method in liquid medium, microdilution, or double dilution method in liquid medium. The third column provides the minimum inhibitory concentration (MIC) and/or minimum fungicidal concentration (MFC) values for each microorganism tested.

FIG. 2 depicts a bar chart comparing the antimicrobial activity of different essential oils at various concentrations. The chart shows five essential oils—peppermint, tea tree, lavender, thyme, and origanum—along the y-axis, with the number of strains shown along the x-axis ranging from 0 to 40. The data is represented by horizontal bars segmented into different concentrations ranging from 256 μg/mL to >4096 μg/mL, as indicated in the legend. The bars are shaded in different grayscale patterns to distinguish between the concentration levels.

FIG. 3 shows a GC/MS analysis table of Melaleuca alternifolia L. essential oil. The table lists 15 active ingredients and their corresponding concentration percentages. The highest concentration component shown is Terpinen-4-ol at 31.62%. The second highest concentration is γ-Terpinene at 15.47%, followed by Terpinolene at 9.05%. Other components include p-Cymene at 4.20%, Isoterpinolene at 3.83%, α-Terpineol at 3.68%, and Leden at 3.62%.

FIG. 4 shows a GC/MS analysis table of Thymus serpyllum L. essential oil. The table lists 15 active ingredients and their corresponding concentration percentages. Carvacrol is shown as the primary active ingredient with a concentration of 59.93%. The second most concentrated ingredient is o-Cymene at 7.67%, followed by γ-Terpinene at 5.94%. Thymol is listed fourth with a concentration of 5.30%.

FIG. 5 shows a table displaying data about the effects of various essential and carrier oils on the survival time of Demodex species. The table contains four columns listing: the type of oil tested, the number of mites used in the testing (n), the mean survival time in minutes (with standard deviation), and the median survival time range in minutes. The table presents data for multiple oil concentrations, including mineral oil as a control and various concentrations of TTO (Tea Tree Oil), BSO, SJWO, SO, and TO.

FIG. 6 illustrates a section view of a mixing arrangement for creating a carvacrol and oil mixture. The arrangement includes the carvacrol liquid 60 and the oregano oil 62 contained within the glass beaker 70. The sonifier 80 extends into the glass beaker 70 for mixing the components. The sonifier 80 is positioned to extend downward into the glass beaker 70 containing the carvacrol liquid 60 and the oregano oil 62.

FIG. 7 shows a section view of the glass beaker 70 containing the carvacrol oil mixture 63. The glass beaker 70 holds the carvacrol oil mixture 63, which appears as a liquid phase in the lower portion of the beaker. The sonifier 80 extends downward into the glass beaker 70, with its tip positioned in the carvacrol oil mixture 63.

FIG. 9 illustrates an orthogonal view showing the application of the carvacrol oil mixture to an eye. The figure depicts the dropper tip 75 positioned above the eye 85. The dropper tip 75 contains the carvacrol oil mixture 63 which is dispensed onto the eye 85 and eyelid margins. The dropper tip 75 is shown oriented vertically above the eye 85 to enable controlled application of the carvacrol oil mixture 63.

FIG. 10 shows an orthogonal view of a dropper vial application system for treating an eye. The figure depicts the dropper vial 90 positioned above the eye 85. The dropper vial 90 contains the carvacrol oil mixture 63 which is dispensed onto the eye 85. The dropper vial 90 is oriented vertically above the eye 85 to allow controlled application of the carvacrol oil mixture 63.

FIG. 11 shows a process for forming a mixture of an antimicrobial agent. The figure illustrates components arranged in a vertical configuration. The carvacrol liquid 60 is shown at the top of the arrangement. Below this is positioned the thymol oil 1162. These components are shown being combined in the glass beaker 70. The sonifier 80 is depicted extending into the glass beaker 70 for mixing the components together. The resulting mixture can have a carvacrol liquid 60 component of 0.5 to 80% depending on the nature of the desired mixture of carvacrol liquid 60 and the selected carrier or essential oil used.

FIG. 12 illustrates a section view of the glass beaker 70 containing the liquid mixture 116. The glass beaker 70 shows the sonifier 80 positioned within the vessel. The sonifier 80 extends downward into the glass beaker 70, with the liquid mixture 116 occupying the lower portion of the beaker. The liquid mixture 116 appears as a dotted pattern within the glass beaker 70, indicating its presence below the sonifier 80.

FIG. 13 shows a kit comprised of carvacrol 1310 and a therapeutic oil 1320 selected from the group consisting of an essential oil, a CBD oil, a cannabis oil, and other cannabis-derived oils, and instructions for use 1330, eye dropper 1340 and a swab 1345.

A number of implementations have been described. Nevertheless, it will be understood that various modifications may be made without departing from the spirit and scope of the disclosure. Accordingly, other implementations are within the scope of the following claims. Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

We claim:

1. A composition for treating eyelid conditions, comprising:
carvacrol; and
a therapeutic oil selected from the group consisting of an essential oil, a CBD oil, a cannabis oil, and other cannabis-derived oils,
wherein the carvacrol and the therapeutic oil are combined to form a mixture.

2. The composition of claim 1, wherein the carvacrol is sonicated in the therapeutic oil.

3. The composition of claim 1, wherein the composition is in a form selected from the group consisting of a cream, a serum, a gel, an ointment, and a lotion.

4. The composition of claim 1, wherein the composition is formulated for topical application to an eyelid.

5. The composition of claim 1, wherein the composition is effective for treating blepharitis.

6. The composition of claim 5, wherein the composition is effective for treating at least one of: chalazion, stye, dry eye syndrome, eyelid dermatitis, and meibomian gland dysfunction.

7. The composition of claim 1, wherein the carvacrol is present in a concentration range of 0.1% to 10% by weight of the composition.

8. A method of treating an eyelid condition, comprising:
applying a composition to an eyelid of a subject, the composition comprising carvacrol and a therapeutic oil selected from the group consisting of an essential oil, a CBD oil, a cannabis oil, and other cannabis-derived oils,
wherein the carvacrol and the therapeutic oil are combined to form a mixture.

9. The method of claim 8, wherein the eyelid condition is blepharitis.

10. The method of claim 9, wherein the eyelid condition further includes at least one of: chalazion, stye, dry eye syndrome, eyelid dermatitis, and meibomian gland dysfunction.

11. The method of claim 8, wherein the composition is applied topically to the eyelid.

12. The method of claim 11, wherein the composition is in a form selected from the group consisting of a cream, a serum, a gel, an ointment, and a lotion.

13. The method of claim 8, wherein the carvacrol is present in a concentration range of 0.1% to 10% by weight of the composition.

14. The method of claim 13, wherein the therapeutic oil is present in a concentration range of 0.1% to 100% by weight of the composition.

15. A kit for treating an eyelid condition, comprising:
a composition comprising carvacrol and a therapeutic oil selected from the group consisting of an essential oil, a CBD oil, a cannabis oil, and other cannabis-derived oils, wherein the carvacrol and the therapeutic oil are combined to form a mixture; and
instructions for use.

16. The kit of claim 15, wherein the composition is in a form selected from the group consisting of a cream, a serum, a gel, an ointment, and a lotion.

17. The kit of claim 15, wherein the composition is effective for treating blepharitis.

18. The kit of claim 17, wherein the composition is effective for treating at least one of: chalazion, stye, dry eye syndrome, eyelid dermatitis, and meibomian gland dysfunction.

19. The kit of claim 15, wherein the carvacrol is present in a concentration range of 0.1% to 10% by weight of the composition.

20. The kit of claim 19, wherein the therapeutic oil is present in a concentration range of 0.1% to 100% by weight of the composition.
